(19) United States
(12) Patent Application Publication
(10) Pub. No.: US 2021/0043311 A1
(43) Pub. Date: Feb. 11, 2021

(54) METHODS AND SYSTEMS FOR MONITORING COMPLIANCE WITH INFECTION PREVENTION PROCEDURES

(71) Applicants: John GRANT, Solana Beach, CA (US); Daniel M. CHAMBERS, Solana Beach, CA (US)

(72) Inventors: John GRANT, Solana Beach, CA (US); Daniel M. CHAMBERS, Solana Beach, CA (US)

(21) Appl. No.: 16/988,644

(22) Filed: Aug. 8, 2020

Related U.S. Application Data
(60) Provisional application No. 62/884,599, filed on Aug. 8, 2019.

(51) Int. Cl. G16H 40/20; G16H 40/67; G16H 10/60; G16H 50/80; G16H 50/30; G16H 50/20

(52) U.S. Cl. CPC G16H 40/20; G16H 40/67; G16H 50/20; G16H 50/80; G16H 50/30; G16H 10/60

(57) ABSTRACT

This invention concerns computer-based methods and systems to monitor and track compliance with infection prevention protocols.

### RELATED APPLICATION

This application claims benefit of and priority to U.S. provisional patent application Ser. No. 62/884,599, filed on 8 Aug. 2019 (attorney docket no. JGT-0100-PV), which is hereby incorporated by reference in its entirety for any all purposes.

### FIELD OF THE INVENTION

The present invention relates to computer-based methods and systems to monitor and track compliance with infection prevention protocols.

### BACKGROUND OF THE INVENTION

### 1. Introduction

The following description includes information that may be useful in understanding the present invention. It is not an admission that any such information is prior art, or relevant, to the presently claimed inventions, or that any publication specifically or implicitly referenced is prior art.

### 2. Background

Information can lead to knowledge, and actionable information can lead to change. It is now easy to collect, measure, and track all kinds of information, including health data, product data, etc. For example, in the healthcare field pathogenic microorganisms such as bacteria can cause infections that can be life-threatening. Specific to healthcare settings, healthcare-associated infections (HAI's) are infections that originate from or occur in a hospital or hospital-like setting. In the U.S., HAI's are estimated to occur in at least 5% of all acute care hospitalizations. The estimated incidence is more than two million cases per year, resulting in significant morbidity, mortality, and expense. Indeed, HAI's are estimated to more than double the mortality and morbidity risks of any admitted patient, and result in about 100,000 deaths a year in the United States alone. Common HAI categories include catheter-related blood stream infections (CRBSI's), surgical site infections, ventilator-associated infections, and urinary catheter infections. In the context of CRBSI's, sites for transmission include the catheters themselves, catheter insertion sites through patients'skin, and other medical devices such as needleless (or needle-free) valves (NACs). The incidence of such infections in patients is increasing, and infection control practitioners (ICP's) often cite non-compliance with known infection prevention guidelines, protocols, regulations, and the like as a cause for HAI's. For instance, improper cleansing of injection sites and NACs are known major causes of CRBSI's. Even with this knowledge, however, straightforward rules and protocols regarding healthcare workers' washing of hands prior to contacting patients are often not followed. Thus, the need remains to provide healthcare workers with efficient tools (e.g., infection prevention devices, protocols, reagents, etc.) to help prevent and/or reduce HAI risk, as well as to provide their supervisors, hospital administrators, etc. with methods and systems to efficiently track, monitor, and facilitate compliance with infection prevention regulations and protocols.

In recent years, electronic medical (or health) records (EMR's and EHR's) have been developed to electronically systematize and store digitized health information of patients and patient populations. Such records can be shared across a range of healthcare settings, typically through network-connected, enterprise-wide information systems or other information networks and exchanges. A patient's EMR can include a range of data, for example, her/his medical history, medication and allergies, immunization status, laboratory test results, radiology images, vital signs, personal statistics such age and weight, and billing information. Today, healthcare providers can use EMR data to improve quality outcomes through care management programs. Furthermore, multiple types of clinical and other data from a patient's EMR can be combined to, for example, assist clinicians in identifying and stratifying chronically ill patients.

EMR systems are generally designed to store data accurately and to capture the state of a patient over time. They also eliminate the need to access earlier paper medical records and assist in ensuring data accuracy, integrity, and legibility as well as reducing the risks of data loss and/or replication since there is only one editable file, and such data is often redundantly backed-up at multiple locations. Moreover, since digital EHR information is searchable and in a single file, EMRs are useful in extracting medical data for analysis of possible trends and long-term changes in a patient's health. EMR's also facilitate population-based studies. To date, however, EMR's and EHR's do not track, or do not interact with other applications that track infection prevention data for patients in healthcare settings.

### DEFINITIONS

Before describing the instant invention in detail, several terms used in the context of the present invention will be defined. In addition to these terms, others are defined elsewhere in the specification, as necessary. Unless otherwise expressly defined herein, terms of art used in this specification will have their art-recognized meanings.

As used herein, the singular forms “a”, “an”, and “the” include plural references unless the context clearly dictates otherwise.

The term “about” refers to approximately a +/−10% variation from the stated value. It is to be understood that such a variation is always included in any given value provided herein, whether or not it is specifically referred to.

The term “and/or”, when used in conjunction with two terms, e.g., A and/or B, means A or B or A and B. When the term is used in a string, e.g., A and/or B, and/or B and C, it means A or B or C, A and B, A and C, B and C, and A, B, and C, and so on for longer strings. Likewise, when a range of values us presented, e.g., 1-10, that range will be understood to include any and all specific values within that range, for example, 3.75 and 7 in the noted 1-10 range, and well as any sub-range, or series of smaller ranges, within the larger range.

An “app” refers to a computer program or application that runs, in whole or in part, on a mobile computing device, e.g., a smartphone (e.g., an iPhone®), laptop computer, tablet computer (e.g., an iPad®, Chromebook®, etc.), etc. In some cases, an app runs in part on a mobile computing device and in part on another computer or computer network (e.g., a cloud-based computer network) linked thereto, for example, via the Internet, be it via a hardwire (e.g., Ethernet) connection or wirelessly.

A “display” is a visual depiction shown on an I/O (input/output) device such as a monitor or touchpad, or within a frame or window depicted by the device. A computer system may “provide a display” of an item or other information, for example, by causing a picture of the item to present on a suitable output device operatively connected to the system, or by sending to an external device (such as a client, smartphone, or the like) a set of data that will enable the device to present the item locally to a user.

The terms “healthcare-associated”, “healthcare-acquired”, “hospital-acquired” “infections”, and the like (or “HAI's”) refer to nosocomial infections patients get while in a healthcare setting to receive treatment for another medical condition. HAI's can occur in any care setting, including hospitals, surgical centers, ambulatory clinics, and long-term care facilities such as nursing homes, rehabilitation facilities, and even properly configured and staffed homes or other private residences.

The term “infection prevention” refers to preventing as well as reducing the risk of contracting an infection, particularly an HAI.

The term “infection prevention regulation” refers to a guideline, law, rule, or regulation promulgated or adopted by a healthcare institution that relates to preventing, reducing the risk of contracting, or, if contracted, reducing the severity of an HAI.

The terms “measure”, “measuring”, “measurement”, and the like refer not only to quantitative measurement of a particular variable but also to qualitative and semi-quantitative measurements. Accordingly, “measurement” also includes detection, meaning that merely detecting a change, without quantification, constitutes measurement.

The term “operably associated” refers to an operable association between two or more components.

A “plurality” means more than one.

A “stakeholder” refers to any interested party, for example, a healthcare provider (e.g., a nurse, a physician, a hospital orderly, etc.), an infection preventionist, an epidemiologist, or a representative of an institution involved in, for example, overseeing, paying or reimbursing for, providing, and/or regulating healthcare delivery to patients.

A “subject” refers to a human subject or human patient.

The terms “cloud computing”, “cloud-based”, “in the cloud”, etc. refer to a computing model, system, or network where services are delivered and used over the Internet on an as-needed basis, relying on shared computing resources rather than local servers or personal computing devices to run applications.

### DESCRIPTION OF THE INVENTION

This invention addresses the shortcomings described above by providing computer-implemented methods and systems to track and monitor the use of healthcare-associated infection (HAI) prevention product(s) and/or protocol(s) that can be easily identified and whose use can be electronically data-logged for real-time or subsequent analysis, report and/or message generation, etc. If desired, information gleaned from such data, alone or in conjunction with other data, may be used, for example, to identify best practices and protocols for HAI prevention and/or risk reduction, to predict if a newly admitted patient has an elevated risk of developing an HAI, etc.

Thus, in one aspect the invention provides computer-implemented infection, preferably HAI, prevention systems. Such systems include a computer or computer network; an electronic record system, e.g., an electronic medical record system that includes at least some of a patient's medical records, that is accessible to the computer or computer network, wherein the electronic record system is at least configured to maintain electronic records for a patient admitted to a healthcare institution, the electronic record system including a first database to store infection prevention data related to use(s) of one or more identifiable infection prevention product(s) and/or protocol(s) in connection with providing care to the patient; an interface to allow input of infection prevention data for patients input, for example, by a healthcare provider and which reflects use of an identifiable infection prevention product or protocol in connection with providing care to patients; and a computer program that configures the computer or computer network to store, maintain, retrieve, and analyze at least a portion of the infection prevention data for at least one patient to determine if care provided to the patient(s) complies with applicable infection prevention regulations. In some embodiments, the system's analysis of infection prevention data occurs in real-time. In others, it occurs retrospectively, and in some of these or other embodiments, the analysis can also be prospective or predictive. For example, real-time analysis can facilitate the system's generation of alerts, warning, or other messages in the event the system determines that a required identifiable infection prevention product and/or protocol had not been used or implemented, while retrospective analyses may allow the identification of “best practices”, be it in terms of infection prevention products to use, infection prevention protocols to implement, etc. Similarly, prospective analysis can facilitate the computer-based identification of factors that will, for example, allow patients having an elevated risk of developing an HAI to be identified at or shortly following admission, thereby allowing implementation of one or more interventions intended to prevent development of an HAI.

The invention envisions that its systems and methods can be adapted to work with any infection prevention product or protocol that can be automatically (via computer) uniquely distinguished or identified, for example, by direct or indirect tagging (or other association) with an identification element such as, for example, a bar code, QR code, or RFID chip. In some embodiments, the identification element (e.g., a barcode) is directly placed (e.g., printed, etched, etc.) on a visible region of a product's surface. In other embodiments, an identification element is indirectly associated with an infection prevention product or protocol, for example, by placing a barcode corresponding to a particular protocol adjacent to a graphical representation of that product or protocol in or on, for example, a computer screen, a laminated placard that displays a chart showing each of several discrete, independently identified infection prevention protocols that can be carried out by healthcare personnel in a particular context, e.g., for example, catheter insertion, push medication administration via a central line or a NAC on an IV set, insertion site dressing change, etc.

Examples of identifiable infection prevention product(s) compatible with and/or protocol(s) include a skin preparation solution and device to disinfect a catheter insertion site prior to catheter insertion, a catheter for vascular access (optionally a peripheral IV catheter, a peripherally inserted central line catheter, or a central line catheter), an insertion site protection device (e.g., Biopatch®), a catheter stabilization device (e.g., Statlock®, tape, etc.), a protective anti-infective film cover for a catheter insertion site ((e.g., a hydrocolloid film such as Tegaderm®, a silicone film such as IV Clear™, etc.), a needlefree access connector (NAC), a disinfecting wipe, a NAC disinfecting device (e.g., SPIN™, Curos®, DualCap®, SwabCap®, etc.), water, disinfecting hand soap, a sterile glove, a face mask, a hair cap, a hand disinfection protocol, a skin preparation protocol, a catheter insertion protocol, a catheter securement protocol, a catheter covering protocol, an extension set-to-catheter connection protocol, a NAC cleansing/disinfection protocol, an IV bag-to-extension set connection protocol, and NAC-based fluid medication administration protocol. In some instances, a protocol may represent a combination of two or more discrete protocols, alone or in combination with one or more infection prevention products, such that identification of, for example, performance of the higher level, more generic protocol signifies performance of each of the identifiable specific protocols included in the more generic identifiable (and identified) protocol.

In some embodiments, the interface wirelessly receives infection prevention data transmitted from a peripheral device, optionally a portable, preferably a handheld or wearable (e.g., worn by a healthcare worker) device, configured to acquire data from or associated with identifiable infection prevention product(s) and/or protocol(s) to allow the system to automatically identify the product and/or protocol.

In some embodiments, the analysis of infection prevention data occurs periodically or in real-time, and wherein the computer or computer network is further optionally configured to, on command and/or periodically, generate a report detailing compliance and/or non-compliance with applicable infection prevention regulations with regard to one, some, or all patients admitted to the healthcare institution. In certain preferred embodiments, the analysis of infection prevention data occurs in real-time, and wherein the computer or computer network is further optionally configured to output to one or more users an alert if the system determines that care being provided to the patient does not comply with applicable infection prevention regulations, wherein the users optionally are orderlies, nurses, infection preventionists, physicians, physician assistants, and hospital administrators. In other preferred embodiments, the analysis of infection prevention data is prospective and results in generating a patient-specific report detailing infection prevention procedures to implement for the patient during an upcoming period, for example, optionally within about the next 6 hours (h), 12 h, 24, 36 h, 48 h, 72 h, 96 h, 120 h, 144 h, or 168 h. Such reports can also cover more than one patients, for example, representing all patients within a given hospital room, department or ward, on a given floor, assigned to a particular healthcare worker during a particular shift, etc.

In some embodiments, the system is configured to allow authenticated user access to infection prevention data stored in the first database, wherein such access optionally allows any authenticated user to view some or all of the infection prevention data stored in the database for one or more patients, optionally via an infection prevention dashboard, on a display, optionally a computer display or the touchscreen on a smartphone.

A related aspect of the invention concerns computer-implemented methods to monitor or track compliance with infection prevention procedures in a healthcare institution such as a hospital, outpatient surgery center, ambulatory care center, etc. Such methods typically employ a computer-implemented infection prevention system to perform a method that includes the steps of (i) providing to a computer or computer network an electronic record system that maintains electronic records for patients admitted to a healthcare institution in a database configured to store infection prevention data related to use(s) of one or more identifiable infection prevention product(s) and/or protocol(s) in connection with providing care to the patients; (ii) via an interface, for each patient at risk of developing an HAI (e.g., a CRBSI), inputting infection prevention data, often by a healthcare provider, reflecting use of an identifiable infection prevention product or protocol in connection with providing care to the patient; and (iii) running a computer program that configures the computer or computer network to store, maintain, retrieve, and/or analyze at least a portion of the infection prevention data for the patient to determine if care provided to the patient complies with applicable infection prevention regulations. In many embodiments, the electronic record system is an EMR or EHR.

The analysis of infection prevention data can occur in real-time or retrospectively, and can relate to a single patient or a group (population) of patients in, for example, the same hospital (or hospital system). When the analysis occurs periodically or in real-time, the computer or computer network can further be configured to, on command and/or periodically, generate a report detailing compliance and/or non-compliance with applicable infection prevention regulations with regard to one, some, or all patients admitted to the healthcare institution. The system can also be further configured to output to one or more users an alert if the system determines that care being provided to a patient does not comply with applicable infection prevention regulations. Such users are preferably authenticated users, and include healthcare personnel such as orderlies, nurses, infection preventionists, physicians, physician assistants, and hospital administrators.

In other embodiments, the system can be configured to allow an authenticated user access to infection prevention data stored in the system's first database, for example, to allow the user to view some or all of the infection prevention data stored in the database (for one or more patients) to be displayed, for example, on an infection prevention dashboard displayed on a computer display or the touchscreen on the user's smartphone.

Another related aspect of the invention concerns computer-implemented methods for identifying an infection prevention product or protocol implemented or used by a healthcare worker from among a plurality of different independently identifiable infection prevention product(s) and/or protocol(s) stored in the system. In general, such methods include using an infection prevention system of the invention to execute a computer program that identifies a particular infection prevention product(s) and/or protocol(s) represented in the system from infection prevention data input into the system by an authenticated user via the interface. Generally, data representing a plurality independently identifiable infection prevention product(s) and/or protocol(s) are stored in a memory used by the computer or computer system thereby allowing the system to identify the infection prevention product or protocol implemented by the user and reflected by the infection prevention data input into the system. Generally, the system determines the identity of a particular infection prevention product or protocol using an identification element tagged thereto or otherwise associated therewith. Identification elements include such things as bar codes, QR codes, and RFID chips, although any suitable device, marking product, or technology (e.g., digital image-based pattern recognition or identification) can be used. Preferably the identification element uniquely identifies the product such that it can be distinguished from other products. It can also include additional identifying information such as batch or lot number, version or revision number, or the like. In some embodiments related to infection prevention product, the identification element can also include serial number information, such that the particular product can be distinguished from among all like products.

In many embodiments, the methods of the invention further include storing the identity of a particular infection prevention product or protocol used in caring for a patient in a database associated with (i.e., a part of or electronically accessible to) the system.

Yet another related aspect of the invention relates to computer-implemented methods for tracking use in a healthcare institution of a plurality of independently identifiable infection prevention products and/or protocols. Such methods generally include using an infection prevention system according to the invention to (i) identify a particular infection prevention product or protocol from infection prevention data input by an authenticated user into the system via the interface from data stored in the system representing a plurality independently identifiable infection prevention product(s) and/or protocol(s), and (ii) store for subsequent use by the system the identity of the particular infection prevention product or protocol in the first database of the system, thereby allowing tracking in the healthcare institution (or healthcare system) of any one or more of the plurality of independently identifiable infection prevention product(s) and/or protocol(s). In such methods, the system of the invention determines the identity of a particular infection prevention product or protocol using an identification element tagged thereto (or another technology that allows the product or protocol type to be distinguished from other infection prevention product or protocol types.

Another aspect of the invention involves computer-implemented methods to manage data stored in an electronic record system that contains infection prevention data related to use of one or more identifiable infection prevention product(s) and/or protocol(s) in connection with providing healthcare to patients. Such methods include using a computer-implemented infection prevention system of the invention to execute a computer program that manages infection prevention data stored in the first database. In some embodiments, management of the infection prevention data stored in one of the system's databases further includes using one or more of the system's computers to perform one or more of the following independently selected functions or tasks: retrieving at least some of the infection prevention data for analysis by the system; displaying at least some of the infection prevention data on a computer display connected to the system; analyzing at least some of the infection prevention data for a particular patient of the healthcare institution to determine if the care provided to the patient complies with applicable infection prevention regulations; transmitting to an computing device of an authenticated user or a computer of the system (i) at least some of the infection prevention data for a patient of the healthcare institution and/or (ii) data representing one or more results of an analysis of some or all of the patient's infection prevention data; integrating patient-specific infection prevention data related to identifiable infection prevention product(s) and/or protocol(s) with an inventory management system and/or an electronic billing system of the healthcare institution; and outputting to one or more authenticated users a message indicating non-compliance with an infection prevention regulation.

Alternative or additional functions or steps can also be performed, including, for example, using the system to determine an HAI risk profile of a patient for whom infection prevention data is stored in the system. In some such embodiments, the method further comprises periodically determining an HAI risk profile for the patient prior to her/his discharge from the healthcare institution. In many of these embodiments, the output of any, preferably some, and even more preferably, all of such HAI risk profile determinations are stored in the system. If the patient's HAI risk profile is determined to be increasing during her/his admission to the healthcare institution, the system can, for example, outputting an alert to at least one authenticated user of such increase and/or output a recommendation for one or more interventions, for example, a clinical intervention (e.g., administration of prophylactic course of antibiotics, a dietary change, the change of one or more infection prevention products (i.e., a disinfecting cap) used in caring for the patient on a more frequent basis than is called for in the manufacturer's use instructions or the corresponding infection prevention protocol, etc.), designed to reduce the patient's HAI risk profile. Other suitable interventions can include more frequent monitoring of the patient, moving the patient to a different floor, ward, or unit, etc. In other preferred embodiments, such methods further include using some or all of the infection prevention data in the system, or from a plurality of such systems each installed at a different healthcare institution to determine infection prevention best practices, be it in terms of infection prevention products, infection prevention protocols, or combinations of infection prevention products, infection prevention protocols.

In a related aspect, the invention involves computer-implemented methods to retrospectively analyze infection prevention data to computationally determine infection prevention best practices. In general such methods include retrospectively computationally analyzing infection prevention data that lacks patient-identifying indicia but that includes HAI occurrence data to determine which identifiable infection prevention product(s) and/or protocol(s) are most effective in reducing HAI occurrence. The absence of patient-identifying indicia in the data serves to protect patient privacy and can result from selecting from patient stored in the system only data fields that lack patient-identifying indicia. In other embodiments, patient data, if present in one more fields selected for analysis, may be masked or otherwise rendered unsuitable for patient identification. In some embodiments, the infection prevention data to be analyzed is collected from a plurality of different healthcare institutions or systems each using an infection prevention system according to the invention. In preferred embodiments of this and other aspects of the invention, such analyses are performed periodically, for example, upon demand of an authenticated user, or automatically on a daily, weekly, monthly, quarterly, semi-annual, or annual basis. Results of such analyses are preferably distributed to one or more stakeholders.

The invention contemplates all combinations of any one or more of the foregoing aspects and/or embodiments, as well as combinations with any one or more of the embodiments set forth in the Representative Embodiments described below.

### REPRESENTATIVE EMBODIMENTS

### Information Flow—Real Time Monitoring

This example describes a representative process by which an authenticated user (e.g., a nurse) can use a handheld barcode scanner to record the exchange of a single-use NAC disinfecting cap that has resided on a NAC for 7 days (or such other period as the cap manufacturer, hospital protocol, or the like has designated as the maximum allowable period a cap can be used to provide NAC protection) after the system determines that the particular NAC disinfecting cap needs to be exchanged. In this example, a hospitalized patient has a peripheral IV that includes at its distal end an intravenous catheter inserted into a vein on the back of one of the patient's hands. The catheter's insertion site is protected by a BioPatch®, the catheter is secured with a Statlock®, and the catheter/insertion site region is covered with Tegaderm®. The proximal end of the catheter is connected to an extension set that includes Y-NAC at its distal end. The luer threads of the “Y” region of the Y-NAC have a disinfecting cap (e.g., a Curos® cap; 3M) that had been screwed on 6 days earlier when the IV was initially connected to the patient in order to passively protect the Y-NAC's injection port immediately after using a sterile alcohol wipe to “scrub the hub” of the Y-NAC. The extension set is connected to an IV line, which line includes two additional Y-NACs to provide additional access ports in the IV, the valve portions of which were also each capped with a passive disinfecting cap (e.g., a Curos® cap) at the same time as the port on the most distal Y-NAC was capped. The proximal end of the IV line is connected to a drip chamber, which is connected to an IV bag container lactated Ringer's solution that is hung from an IV pole adjacent to the patient's bed. Also hanging from the pole is a strip of ready-to-use passive disinfecting caps. The strip includes a barcode that can be scanned with a handheld barcode reader each time a cap is removed from the strip. As will be appreciated, any suitable barcode configuration (or other data-encoding system that allows automatic data logging of at least product type information) can be used. Here, the strip includes a barcode next to each ready-to-use cap disposed on the strip. In other embodiments, the strip can include just one barcode, which can be scanned each time a protective cap is removed from the strip for attachment to NAC.

As is common for NAC disinfecting caps, the protective caps typically have a maximum dwell period of seven days, meaning that for each of them, if it is not removed within seven days after being screwed onto a NAC, it must be removed and replaced with a new protective cap. In this example, when each of these three caps is removed from the strip and initially screwed onto a corresponding Y-NAC as the IV is being initially established, the nurse doing so would use a handheld barcode scanner to scan the barcode. That data, including the date and time of the scanning operation, is automatically uploaded into the system, creating a record closely approximating when each cap is removed from the strip and, assuming that the particular cap, after being removed the strip, is immediately screwed onto a Y-NAC port that had just been cleaned using, for example, a sterile alcohol wipe. If desired, a hospital procedure can dictate that the nurse also, at the time a protective cap is screwed onto a NAC, scan a barcode (or other suitable AIDC (Automatic Identification and Data Capture) technology) to indicate that a “scrub the hub” protocol had been used immediately prior to placing the cap on the corresponding NAC port. Alternatively, if the packaging for the alcohol wipe used to perform the “scrub the hub” procedure on the NAC port includes a suitable barcode (or other AIDC technology), scanning (or otherwise detecting and uploading into the system) that code upon opening the wipe package within a predefined period of also scanning a protective cap barcode can be used to track in the system not only use of an alcohol wipe but also that wipe's use as a proxy to indicate that the port to which the cap is then attached has been appropriately “scrubbed” just prior to the cap's attachment. In any event, if the system detects (via absence in real-time of data to otherwise indicate) that a protective cap has been attached to a NAC port that, for example, has not first been scrubbed in accordance with the applicable “scrub the hub” protocol, the system could automatically generate a warning, alert, or other message that could be immediately sent, for example, to the nurse's smartphone, an in-room computer display, supervisor, etc. Absent the prompt uploading into the system of data to indicate that corrective action(s) has been taken, the system could generate and send additional alerts, warnings, or other messages to other hospital personnel (e.g., an infection prevention specialist, nurse supervisor, etc.).

Returning to use of the system to track expiration, change-out, or other such dates for taking action, particularly to replace a protective cap (or other device or tool) nearing the end of its maximum permissible “dwell” period, periodically the system will assess whether a particular device being tracked by the system is within or approaching preset operating parameters (e.g., maximum dwell time). As an example, the system can be programmed to send one or more an alerts to appropriate hospital personnel as a protective cap (or group of protective caps, e.g., all protective caps on a particular peripheral IV on a particular patient) is approaching that particular cap's maximum dwell time, for example, 24 hours before the end of a preset 168 hr. maximum dwell time. If desired, additional alerts or messages to the same (and/or different) personnel can be sent in the event the system is not updated within a preset period, for example, 4-6 hr., to indicate that the protective cap(s) nearing the end of its(their) maximum dwell period have been replaced with new protective caps (preferably after the corresponding NAC port having also been “scrubbed” again with a sterile alcohol wipe, which data the system preferably also monitors).

### Information Flow—Retrospective Monitoring

As described above, the systems and methods of the invention can be used for retrospective monitoring of infection prevention data, be it on a patient-by-patient or patient population basis.

### Information Flow—Predictive Analytics

As described above, the systems and methods of the invention can also be used prospectively. For example, a neural network can be used to analyze a large multivariate dataset to identify variables that suggest that a patient outside the training dataset is at higher risk for developing an HAI and should thus, for example, be more closely monitored, put on antibiotics, etc.

As is known, all hospitalized patients are at risk for, or susceptible to, contracting an HAI. Some patients are inherently at greater risk for contracting such infections, including neonates, infants, and young children, the elderly, and immune-compromised patients. Other well-known HAI risk factors include the length of admission (longer periods of hospital admission strongly correlate with increased HAI incidence), the use of indwelling catheters (e.g., central lines, peripheral IV's, and urinary catheters), failure by healthcare personnel to wash their hands or observe other hygiene protocols, and high rates of antibiotic use (which can lead to the development of antibiotic drug resistance in pathogenic microorganisms).

### Web-Based Implementation

It will be understood that individual devices useful in connection with the invention (e.g., smartphones, tablet computers, other I/O devices, etc.) are preferably part of a larger, cloud-based system that delivers services over the Internet on an as-needed basis, relying on shared computing resources rather than local servers or personal computing devices to run applications. Preferably, an app is used to configure a computer that is part of a data logging device (e.g., a handheld barcode scanner) or other mobile computing device, e.g., a smartphone (e.g., an Apple iPhone®, Samsung Galaxy mobile phone), laptop computer, tablet computer (e.g., an iPad®, Chromebook®, etc.) to perform a method of the invention, for example, to scan a barcode associated with an infection prevention product (e.g., a NAC disinfecting cap, a catheter securement device, etc.) or protocol (e.g., a hand-washing protocol, a skin preparation protocol, etc.) computationally determine from image data captured using a camera associated with (e.g., integrated as part of) the device whether one or more blood flow parameters (e.g., HRV, HR, etc.) associated or correlated with psychological stress detected in blood perfusing regions of interest in the skin of a subject, alone or together with other data correlated with acute psychological stress, indicate that the subject was experiencing acute psychological stress at the time the image data was collected. In some embodiments, an app runs in part on a mobile computing device and in part on another computer or computer network (e.g., a cloud-based computer network) linked thereto, for example, via the Internet, be it via a hardwire (e.g., Ethernet) connection or wirelessly.

In some embodiments, a cloud-based system according to the invention will include at least one processor, read only memory (ROM), random access memory (RAM), one or more network interfaces, one or more mass storage devices, and one or more databases coupled together by a communications bus. Processor(s) can be microprocessors, and the mass storage device can comprise one or more local or remote magnetic disk drives, solid-state storage devices, and/or storage devices. Mass storage device (or interconnected system comprised of a plurality of such devices) and memory modules are connected to the processor(s) to allow the processor(s) to write data into and read data from the storage and memory devices. Network interfaces couple the system to a network, for example, the Internet. The nature of the network and of the devices that may be interposed between the system and the network determines the kind of network interface used in the system. In some embodiments, for example, the network interface may be an Ethernet interface that connects the system to a local area network, which, in turn, connects to the Internet. The network also preferably includes a cellular interface that to provide connectivity between the network and users' mobile computing devices (e.g., smartphones, laptop personal computers, tablet computers, etc.), although users can also employ desktop and other computers having suitable data I/O devices operably associated therewith. Associated databases can be used for organizing and storing data that may be needed or desired in performing the method steps on the invention, including storing infection prevention product and/or protocol data obtained from, for example, handheld barcode scanners (and/or other sensors useful to collect the desired data) used by doctors, nurses, and other infection prevention specialists. In some embodiments, the processor(s) and the mass storage device(s) can be configured to perform the functions of the database.

As is known, processors can be configured to read and execute program code instructions stored in machine-readable storage devices. Under control of program code, in some embodiments the processors configure the system and/or users' corresponding app-running devices (e.g., smartphones) to function in conjunction with the systems and the methods of the invention. The program code instructions may be embodied in other machine-readable storage media, such as additional hard drives, floppy diskettes, CD-ROMs, DVDs, flash memories, and similar devices. The program code can also be transmitted over a transmission medium, for example, over electrical wiring or cabling, through optical fiber, wirelessly, or by any other form of physical or wireless transmission. The transmission can take place over a dedicated link between telecommunication devices, or through a wide- or local-area network, such as the Internet, an intranet, extranet, or any other kind of public or private network.

Although the process steps and decisions (if decision blocks are present) may be described serially herein, certain steps and/or decisions can be performed by separate elements in conjunction or in parallel, asynchronously or synchronously, in a pipelined manner, or otherwise. There is no particular requirement that the steps and decisions be performed in the same order in which this description lists them, except where a specific order is inherently required, explicitly indicated, or is otherwise made clear from the context. Furthermore, not every illustrated step and decision block may be required in every embodiment in accordance with the concepts described in this document, while some steps and decision blocks that have not been specifically illustrated may be desirable or necessary in some embodiments in accordance with the concepts. It should be noted, however, that specific embodiments/variants/examples use the particular order(s) in which the steps and decisions (if applicable) are shown and/or described.

In various embodiments, the features, elements, and limitations of apparatus and processes described throughout this document can be present individually, or in any combination or permutation, except where the presence or absence of specific feature(s)/element(s)/limitation(s) is inherently required, explicitly indicated, or otherwise made clear from the context.

Unless the context clearly requires otherwise, throughout the description above and the appended claims, the words “comprise”, “comprising”, and the like are to be construed in an inclusive sense as opposed to an exclusive or exhaustive sense; that is to say, in a sense of “including, but not limited to”. Words using the singular or plural number also include the plural or singular number, respectively. Additionally, the words “herein”, “hereunder”, “above”, “below”, and words of similar import refer to this application as a whole and not to any particular portions of this application. When the word “or” is used in reference to a list of two or more items, that word covers all of the following interpretations of the word: any of the items in the list; all of the items in the list; and any combination of the items in the list.

The foregoing description, for purpose of explanation, has been described with reference to specific embodiments. However, the illustrative discussions above are not intended to be exhaustive or to limit the invention to the precise forms disclosed. Many modifications and variations are possible in view of the above descriptions. The embodiments were chosen and described in order to best explain the principles of the invention and its practical applications to thereby enable others skilled in the art to best utilize the invention and various embodiments with various modifications as are suited to the particular use contemplated. As such, the invention extends to all functionally equivalent structures, methods, and uses, such as are within the scope of the appended claims, and it is intended that the invention be limited only to the extent required by the applicable rules of law.

With regard to the appended claims, it is understood that, once issued, they represent only patentable subject matter, and any claimed process, machine, or article of manufacture according to the invention means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically exclude any unpatentable embodiment(s). Also, the claims appended hereto are to be interpreted both to provide the broadest reasonable scope, as well as to preserve their validity. Furthermore, if one or more of the statutory requirements for patentability are amended or if the standards change for assessing whether a particular statutory requirement for patentability is satisfied from the time this application is filed or issues as a patent to a time the validity of one or more of the appended claims is questioned, the claims are to be interpreted in a way that, to the maximum extent then permissible, (1) preserves their validity and (2) provides the broadest reasonable interpretation under the circumstances.

What is claimed is:

1. A computer-implemented infection prevention system, comprising:
a. a computer or computer network;
b. an electronic record system accessible to the computer or computer network, wherein the electronic record system is configured to at least maintain electronic records for a patient admitted to a healthcare institution, the electronic record system including a first database to store infection prevention data for a patient related to use(s) of one or more identifiable infection prevention product(s) and/or protocol(s) in connection with providing care to the patient;
c. an interface to allow input of infection prevention data for patients, optionally input by a healthcare provider, reflecting use of an identifiable infection prevention product or protocol in connection with providing care to the patient; and
d. a computer program that configures the computer or computer network to store, maintain, retrieve, and analyze at least a portion of the infection prevention data for at least one patient to determine if care provided to the patient(s) complies with applicable infection prevention regulations.

2. A system according to claim 1 wherein:
a. the electronic record system is an electronic medical record system that includes medical records for the patient; and/or
b. the analysis of infection prevention data occurs in real-time or retrospectively; and/or
c. the interface wirelessly receives infection prevention data transmitted from a peripheral device configured to acquire data from or associated with an identifiable infection prevention product to allow its identification by the system, wherein the peripheral device optionally is portable and optionally is a handheld device or a device worn by a healthcare worker; and/or
d. the analysis of infection prevention data occurs periodically or in real-time, and wherein the computer or computer network is further optionally configured to, on command and/or periodically, generate a report detailing compliance and/or non-compliance with applicable infection prevention regulations with regard to one, some, or all patients admitted to the healthcare institution; and/or
e. the analysis of infection prevention data occurs in real-time, and wherein the computer or computer network is further optionally configured to output to one or more users an alert if the system determines that care being provided to the patient does not comply with applicable infection prevention regulations, wherein the users optionally are orderlies, nurses, infection preventionists, physicians, physician assistants, and hospital administrators; and/or
f. the analysis of infection prevention data is prospective and results in generating a patient-specific report detailing infection prevention procedures to implement for the patient during an upcoming period, optionally within the next 6 h, 12 h, 24, 36 h, 48 h, 72 h, 96 h, 120 h, 144 h, or 168 h; and/or
g. the system is configured to allow authenticated user access to infection prevention data stored in the first database, wherein such access optionally allows an authenticated user to view some or all of the infection prevention data stored in the database for one or more patients to be displayed, optionally via an infection prevention dashboard, on a display, optionally a computer display or the touchscreen on a smartphone.

3. A system according to claim 1 wherein the identifiable infection prevention product(s) and/or protocol(s) is(are) selected from the group consisting of a skin preparation solution to disinfect a catheter insertion site prior to catheter insertion, a catheter for vascular access (optionally a peripheral IV catheter, a peripherally inserted central line catheter, or a central line catheter), an insertion site protection device (optionally Biopatch®), a catheter stabilization device (optionally Statlock®), a protective anti-infective film cover for a catheter insertion site ((optionally a hydrocolloid film such as Tegaderm® or a silicone film such as IV Clear™), a needlefree access connector (NAC), a disinfecting wipe, a NAC disinfecting device (optionally SPIN™, Curos®, DualCap®, SwabCap®, etc.), water, disinfecting hand soap, a sterile glove, a face mask, a hair cap, a hand disinfection protocol, a skin preparation protocol, a catheter insertion protocol, a catheter securement protocol, a catheter covering protocol, an extension set-to-catheter connection protocol, a NAC cleansing/disinfection protocol, an IV bag-to-extension set connection protocol, and NAC-based fluid medication administration protocol.

4. A system according to claim 3 wherein the identifiable infection prevention product(s) and/or protocol(s) is(are) is tagged with an identification element, optionally a bar code, QR code, or RFID chip, that allows it to be identified automatically.

5. A computer-implemented method to selected from the group consisting of:
5.1. a computer-implemented method to monitor compliance with infection prevention procedures in a healthcare institution, optionally a hospital, an outpatient surgery center, or an ambulatory care center, comprising using a computer-implemented infection prevention system to perform a method comprising the steps of:
a. providing to a computer or computer network an electronic record system that maintains electronic records for patients admitted to a healthcare institution in a database configured to store infection prevention data related to use(s) of one or more identifiable infection prevention product(s) and/or protocol(s) in connection with providing care to the patients;
b. via an interface, for each patient at risk of developing a healthcare-associated infection (HAI), optionally a catheter-related bloodstream infection, inputting infection prevention data, optionally by a healthcare provider, reflecting use of an identifiable infection prevention product or protocol in connection with providing care to the patient; and
c. running on the computer or computer network a computer program that configures the computer or computer network to store, maintain, retrieve, and analyze at least a portion of the infection prevention data for the patient to determine if care provided to the patient(s) complies with applicable infection prevention regulations;
5.2. a computer-implemented method for identifying an infection prevention product or protocol from among a plurality of different independently identifiable infection prevention product(s) and/or protocol(s), the method comprising using an infection prevention system according to claim A1 to execute a computer program that identifies a particular infection prevention product(s) and/or protocol(s) from infection prevention data input by an authenticated user into the system via the interface from a plurality independently identifiable infection prevention product(s) and/or protocol(s) stored in a memory used by the computer or computer system, thereby identifying the infection prevention product or protocol;
5.3. a computer-implemented method for tracking use in a healthcare institution of a plurality of independently identifiable infection prevention product(s) and/or protocol(s), the method comprising using an infection prevention system according to claim A1 to:
a. identify a particular infection prevention product or protocol from infection prevention data input by an authenticated user into the system via the interface from data stored in the system representing a plurality independently identifiable infection prevention product(s) and/or protocol(s); and
b. store for subsequent use by the system the identity of the particular infection prevention product or protocol in the first database of the system, thereby allowing tracking in the healthcare institution of any one or more of the plurality of independently identifiable infection prevention product(s) and/or protocol(s);
5.4. a computer-implemented method of managing data stored in an electronic record system holding infection prevention data related to use of one or more identifiable infection prevention product(s) and/or protocol(s) in connection with providing healthcare to patients, comprising using a computer-implemented infection prevention system according to claim A1 to execute a computer program that manages infection prevention data stored in the first database; and
5.5. a computer-implemented method of retrospectively analyzing infection prevention data to computationally determine infection prevention best practices, the method comprising retrospectively computationally analyzing infection prevention data that lacks patient-identifying indicia but that includes HAI occurrence data to determine which identifiable infection prevention product(s) and/or protocol(s) are most effective in reducing HAI occurrence.

6. A method according to claim 5.1 wherein:
a. the electronic record system is an electronic medical record system that includes medical records for patients; and/or
b. the analysis of infection prevention data occurs in real-time or retrospectively; and/or
c. the identifiable infection prevention product(s) and/or protocol(s) is(are) selected from the group consisting of a skin preparation solution to disinfect a catheter insertion site prior to catheter insertion, a catheter for vascular access (optionally a peripheral IV catheter, a peripherally inserted central line catheter, or a central line catheter), an insertion site protection device (optionally Biopatch®), a catheter stabilization device (optionally Statlock®), a protective anti-infective film cover for a catheter insertion site ((optionally a hydrocolloid film such as Tegaderm® or a silicone film such as IV Clear™), a needlefree access connector (NAC), a disinfecting wipe, a NAC disinfecting device (optionally SPIN™, Curos®, DualCap®, SwabCap®, etc.), water, disinfecting hand soap, a sterile glove, a face mask, a hair cap, a hand disinfection protocol, a skin preparation protocol, a catheter insertion protocol, a catheter securement protocol, a catheter covering protocol, an extension set-to-catheter connection protocol, a NAC cleansing/disinfection protocol, an IV bag-to-extension set connection protocol, and NAC-based fluid medication administration protocol, wherein the identifiable infection prevention product(s) and/or protocol(s) optionally is(are) is tagged with an identification element, optionally a bar code, QR code, or RFID chip, that allows it to be identified automatically; and/or
d. the interface wirelessly receives infection prevention data transmitted from a peripheral device configured to acquire data from or associated with an identifiable infection prevention product to allow its identification by the system, wherein the peripheral device optionally is portable and optionally is a handheld device or a device worn by a healthcare worker; and/or
e. the analysis of infection prevention data occurs periodically or in real-time, and wherein the computer or computer network is further optionally configured to, on command and/or periodically, generate a report detailing compliance and/or non-compliance with applicable infection prevention regulations with regard to one, some, or all patients admitted to the healthcare institution; and/or
f. the analysis of infection prevention data occurs in real-time, and wherein the computer or computer network is further optionally configured to output to one or more users an alert if the system determines that care being provided to the patient does not comply with applicable infection prevention regulations, wherein the users optionally are orderlies, nurses, infection preventionists, physicians, physician assistants, and hospital administrators; and/or
g. the analysis of infection prevention data is prospective and results in generating a patient-specific report detailing infection prevention procedures to implement for the patient during an upcoming period, optionally within the next 6 h, 12 h, 24, 36 h, 48 h, 72 h, 96 h, 120 h, 144 h, or 168 h; and/or
h. the system is configured to allow authenticated user access to infection prevention data stored in the first database, wherein such access optionally allows an authenticated user to view some or all of the infection prevention data stored in the database for one or more patients to be displayed, optionally via an infection prevention dashboard, on a display, optionally a computer display or the touchscreen on a smartphone.

7. A method according to claim 5.2 wherein:
a. the system determines the identity of a particular infection prevention product(s) and/or protocol(s) is(are) using the identification element tagged to the infection prevention product(s) and/or protocol(s), wherein the identification element optionally is a bar code, QR code, or RFID chip: and/or
b. the method further comprises storing the identity of the particular infection prevention product or protocol in the first database of the system.

8. A method according to claim 5.3 wherein:
a. the method determines the identity of a particular infection prevention product(s) and/or protocol(s) is(are) using the identification element tagged to the infection prevention product(s) and/or protocol(s), wherein the identification element optionally is a bar code, QR code, or RFID chip: and/or
b. the method determines the identity of a particular infection prevention product(s) and/or protocol(s) is(are) using the identification element tagged to the infection prevention product(s) and/or protocol(s), wherein the identification element optionally is a bar code, QR code, or RFID chip.

9. A method according to claim 5.4 wherein:
a. the method further comprises using the system to determine an HAI risk profile of a patient for whom infection prevention data is stored in the system; and/or
b. the method further comprises periodically determining an HAI risk profile for the patient prior to her/his discharge from the healthcare institution, optionally storing the output of each such HAI risk profile determination in the system, and, if the patient's HAI risk profile increases during her/his admission to the healthcare institution, optionally (i) outputting an alert to at least one authenticated user of such increase and/or (ii) outputting a recommendation for one or more interventions, optionally clinical intervention(s), designed to reduce the patient's HAI risk profile; and/or
c. the method further comprises using some or all of the infection prevention data to determine infection prevention best practices.

10. A method according to claim 5.4 wherein management of the infection prevention data stored in the first database comprises using one or more of the system's computers to perform at least one of the following:
a. retrieving at least some of the infection prevention data for analysis by the system;
b. displaying at least some of the infection prevention data on a computer display connected to the system;
c. analyzing at least some of the infection prevention data for a particular patient of the healthcare institution to determine if the care provided to the patient complies with applicable infection prevention regulations;
d. transmitting to an computing device of an authenticated user or a computer of the system (i) at least some of the infection prevention data for a patient of the healthcare institution and/or (ii) data representing one or more results of an analysis of some or all of the patient's infection prevention data;
e. integrating patient-specific infection prevention data related to identifiable infection prevention product(s) and/or protocol(s) with an inventory management system and/or an electronic billing system of the healthcare institution; and
f. outputting to one or more authenticated users a message indicating non-compliance with an infection prevention regulation.

11. A method according to claim 5.5 wherein:
a. the infection prevention data to be analyzed is collected from a plurality of different healthcare institutions or systems each using an infection prevention system according to claim 1; and/or
b. the method is performed periodically, optionally on demand, daily, weekly, monthly, quarterly, semi-annually, or annually; and/or
c. the results of the analysis are distributed to one or more stakeholders.
